(19) United States
(12) Patent Application Publication
(10) Pub. No.: US 2026/0056176 A1
(43) Pub. Date: Feb. 26, 2026

(54) DETECTION OF TVOCs, OZONE AND NOx CONCENTRATIONS USING A METAL-OXIDE GAS SENSOR ARRAY

(71) Applicant: Renesas Electronics America Inc., Milpitas, CA (US)

(72) Inventors: Ravi Kanth Reddy CHILUMULA, Erie, CO (US); Anna Schröter, Dresden (DE); Christian MEYER, Dresden (DE); Clayton John KOSTELECKY, Longmont, CO (US); David SIMPSON, Vero Beach, FL (US); Holger SAALBACH, Grenzstrasse (DE); Ronald Uwe SCHREIBER, Grenzstrasse (DE)

(73) Assignee: Renesas Electronics America Inc., Milpitas, CA (US)

(21) Appl. No.: 18/889,585

(22) Filed: Sep. 19, 2024

Related U.S. Application Data
(60) Provisional application No. 63/685,914, filed on Aug. 22, 2024.

(51) Int. Cl. G01N 33/00; G01N 27/04

(52) U.S. Cl. CPC G01N 33/0034; G01N 27/048; G01N 33/0037; G01N 33/0039; G01N 33/0047

(57) ABSTRACT

An apparatus is provided that includes one or more first MOx sensors, one or more second MOx sensors, and one or more humidity and/or temperature sensors configured to detect, identify, and quantify a first gas, a second gas, and a third gas, where the first gas is a TVOC, the second gas is NO₂, and the third gas is O₃. At least one processor can be configured to receive resistance data, humidity data and temperature data from the one or more first MOx sensors, the one or more second MOx sensors, and the one or more humidity and/or temperature sensors. The at least one process can run a pre-trained or continuously learning neural network, or other machine learning models, to detect, identify, and quantify the first gas, the second gas, and the third gas, while mitigating humidity, temperature, and O₃ influence on the overall output performance.

### CROSS-REFERENCE TO RELATED APPLICATION

The present application claims the benefit of U.S. Patent Application No. 63/685,914, titled “DETECTION OF TVOCs, OZONE AND NOx CONCENTRATIONS USING A METAL-OXIDE GAS SENSOR ARRAY” and filed on Aug. 22, 2024, which is hereby incorporated by reference in its entirety.

### TECHNICAL FIELD

Embodiments of the present disclosure are related to the use of an array of sensors to detect specific gases and, in particular, to systems and methods to quantify the presence of these specific gases.

### BACKGROUND

With increased awareness to the importance of indoor air quality and its impact on human productivity and long-term health effects, there is a need to develop low cost, low power, selective smart sensors that can accurately detect, identify, and quantify the gases of importance. There are several EPA studies that mention both Total Volatile Organic Compounds (TVOCs) and Total Oxidant Air Pollutants (Ozone and NOx) as the majority contributors to indoor air pollution along with dust particles, carbon monoxide, carbon dioxide, etc.

MOx sensors have been widely considered for gas detection, identification, and quantification because they provide cost, size and power benefits. MOx sensors typically consist of a substrate, heater, and a metal oxide surface. More recently for digital sensors an application-specific integrated circuit (ASIC) is designed for heater control and resistance read out. To obtain readable and useful resistance signals out of MOx sensors, they are typically heated at temperatures between 150° C. and 400° C. Various algorithmic and machine learning techniques are then used to convert the raw resistance data into user friendly outputs which indicate the specific gas and amount of gas concentration in sampled air. Along with MOx sensors, a humidity and temperature sensor is commonly utilized to mitigate the cross-interference of both humidity and temperature on the gas measurement outputs.

### SUMMARY

According to an embodiment, an apparatus is provided. The apparatus comprises one or more first metal-oxide (MOx) sensors configured to detect or respond to a first gas and the third gas, wherein the first gas is a total volatile organic compound (TVOC) and third gas is Ozone(O₃) and one or more second MOx sensors configured to detect or respond to a second gas and a third gas wherein the second gas is nitrogen dioxide (NO₂) and the third gas is Ozone (O₃). The apparatus further comprises at least one processor configured to receive resistance data from the one or more first MOx sensors and the one or more second MOx sensors to detect, identify, and quantify the first gas, the second gas, and the third gas; after determining if there is O₃ present utilizing a detection result from the resistance data of the one or more of the first MOx sensor, and one or more of the second MOx sensor; and to adapt the baseline estimation process and further based on the detection result, determine whether the one or more second MOx sensors quantify the second gas or the third gas.

In a first implementation of this embodiment, the apparatus includes one or more humidity and temperature (RH/T) sensors to detect and measure the humidity and temperature of the air.

In a second implementation of this embodiment, the one or more first MOx sensors, the one or more second MOx sensors, and the one or more RH/T sensors are organized in one or more sensor arrays, where each of the one or more sensor arrays comprise one of each the first MOx sensor, the second MOx sensor, and the RH/T sensor.

In a third implementation of this embodiment, the at least one processor is further configured to execute the instructions in response to an increase in the detection result from the resistance data of the one or more first MOx sensors increasing above a first predetermined range and the resistance data of the one or more second MOx sensors increasing above a second predetermined range to determine that O₃ is present.

In a fourth implementation of this embodiment, the at least one processor is further configured to execute the instructions in response to an increase in the detection result from the resistance data of the one or more first MOx sensors staying within a first predetermined range and the resistance data of the one or more second MOx sensors increasing above a second predetermined range, determining that NO₂ is present.

In a fifth implementation of this embodiment, the at least one processor is further configured to execute the instructions in response to an increase in the detection result from the resistance data from the one or more first MOx sensors decreasing below a first predetermined range and the resistance data of the one or more second MOx sensors staying within a second predetermined range, determining that volatile organic compounds (VOCs) are present.

In a sixth implementation of this embodiment, the at least one processor is further configured to execute the instructions in response to an increase in the detection result from the resistance data of the one or more MOx sensors increasing above a first predetermined range and the resistance data of the one or more second MOx sensors staying within a second predetermined range, determining that the air is clean.

In a seventh implementation of this embodiment, the at least one processor is further configured to execute the instructions to store and/or retrieve previously determined baseline values for the one or more first MOx sensors and the one or more second MOx sensors, and the one or more RH/T sensors.

In an eighth implementation of this embodiment, the at least one processor is further configured to execute the instructions of comparing the previously determined baseline values for the one or more first MOx sensors and the one or more second MOx sensors to the detection result from the resistance data received from the one or more first MOx sensors and the resistance data received from the one or more second MOx sensors.

In a ninth implementation of this embodiment, the at least one processor is further configured to execute the instructions quantifying the amount of TVOC, O₃, and NO₂ in response to the detection result being compared to the previously determined baseline values for the one or more first MOx sensors, the one or more second MOx sensors and the one or more RH/T sensors.

According to another embodiment, a method for sensing gas in air is provided. The method comprises quantifying, by one or more processors and one or more first metal-oxide (MOx) sensors, a first gas that is a total volatile organic compound (TVOC); quantifying, by the one or more processors and one or more second MOx sensors, a second gas and a third gas, where the second gas and third gas is either nitrogen dioxide (NO₂) or Ozone (O₃) respectively, receiving, by the one or more processors, resistance data from the one or more first MOx sensors and the one or more second MOx sensors; and based on the O₃ presence detection result, determining, by on the one or more processors, whether the one or more second MOx sensors quantify the second gas or the third gas.

According to yet another embodiment, at least one non-transitory computer-readable storage medium that stores a computer-executable program comprises instructions which, when configured to be executed by a computer, cause the computer to execute the processing of detecting, identifying, and quantifying, by one or more first metal-oxide (MOx) sensors a first gas that is a total volatile organic compound (TVOC); detecting, identifying, and quantifying, by one or more second MOx sensors, a second gas and a third gas, where the second gas and third gas are either nitrogen dioxide (NO₂) or Ozone (O₃) respectively receiving resistance data from the one or more first MOx sensors and the one or more second MOx sensors; and based on the O₃ presence detection result, determining whether the one or more second sensors quantify the second gas or the third gas.

These and other embodiments are discussed below with respect to the following figures.

### BRIEF DESCRIPTION OF THE DRAWINGS

FIGS. 1A and 1B illustrate exemplary systems for an apparatus that quantifies gas in the air, according to embodiments of the present disclosure;

FIG. 2 is a system level block diagram that illustrates the inputs and outputs, according to embodiments of the present disclosure;

FIG. 3 illustrates a logic table to show how the increase or decrease of the signals from both the first MOx sensor and the second MOx sensor are used to determine which gas is present in the air, according to embodiments of the present disclosure;

FIG. 4A illustrates the typical raw responses of the first and second MOx sensors to TVOC exposures, O₃ exposures, and NO₂ exposures, according to embodiments of the present disclosure;

FIG. 4B illustrates the typical raw responses from a humidity sensor and a temperature sensor, specified as percent humidity (% RH) and degrees Celsius (° C.), according to embodiments of the present disclosure;

FIG. 5A illustrates an exemplary flow chart of a technique for detecting, identifying, and quantifying multiple gases in the air with built-in humidity and temperature compensation and O₃ compensation, according to embodiments of the present disclosure;

FIG. 5B illustrates an exemplary flow chart of a technique for dynamically estimating baseline values using one or more MOX sensor resistances and avoiding the influence of Ozone on outputs, according to embodiments of the present disclosure; and

FIG. 6 is a flow diagram of a method to quantify gas in the air, according to embodiments of the present disclosure.

### DETAILED DESCRIPTION

Metal oxide sensors have significant benefits, in terms of cost, power, size etc., however, they are not selective to individual gases. As an example, an Ozone (O₃)/Nitrogen Dioxide (NO₂) sensor can respond to either gas but cannot specify which gas is actually being detected. Similarly, TVOC sensors usually respond to O₃ as well which results in baseline estimation cross-interference and inaccurate TVOC quantification. Therefore, there is a need for the development of sensors that can selectively detect, identify, and quantify individual gases, while at the same time being able to mitigate the cross interference of each gas with the other gases. Accordingly, the present disclosure provides a solution for detecting individual gases and avoiding cross interference.

Embodiments of the present disclosure provide a solution to the above identified deficiencies for being able to detect individual gases, particularly Ozone (O₃) and Nitrogen Dioxide (NO₂) gases. The solution involves systems and methods that include one or more sensors and/or sensor arrays. The one or more sensors and/or sensor arrays may include one or more TVOC/O₃ sensors and one or more O₃/NO₂ sensors, to detect the presence of O₃ and NO₂ in the air. Additionally, one or more humidity and temperature (RH/T) sensors may be used in combination with the one or more TVOC/O₃ sensors and the one or more O₃/NO₂ sensors. The one or more RH/T sensors, the one or more TVOC/O₃ sensors, and the one or more O₃/NO₂ sensors may be incorporated into one or more sensor arrays with each sensor array including a RH/T sensor, a TVOC/O₃ sensor, and an O₃/NO₂ sensor.

In some embodiments of the present disclosure, the TVOC/O₃ sensor may be a sensor configured to detect TVOCs, estimate CO₂, and monitor indoor air quality and the O₃/NO₂ sensor may be a sensor configured to detect O₃/NO₂ and measure MOx resistance. In such embodiments, the one or more first sensors may be sensors and the one or more second sensors may be sensors. In either case, the present disclosure refers to the one or more first sensors being TVOC/O₃ sensors and the one or more second sensors being O₃/NO₂ sensors. However, in other embodiments the one or more first sensors may be O₃/NO₂ sensors (e.g., the sensors) and the one or more second sensors may be the TVOC/O₃ sensors (e.g., the sensors).

The present disclosure utilizes one or more smart operating methods that involve multi-temperature sweeps to heat the one or more MOx sensors to obtain an array of sensor resistance data and also other parameters representing sensor internal operation conditions. The MOx sensors can be used as smart sensors to detect, or to respond to, at least one of TVOCs, O₃ and NOx. The temperature sweeps involve an active heating mode at various temperatures and a passive mode where the heater is turned OFF. The systems and methods described herein can also address automatic drift compensation, output stability, ambient compensation, etc. are also disclosed as a part of the present disclosure. The system and methods described herein are designed for real-time and/or post-processing use cases and provide outputs for every sample and are built to scale. These systems and methods involve automatic baseline methods that adapt dynamically to identify clean air and use it as a reference to derive a normalized signal, in which the strength of the signal is directly correlated to a gas concentration. The systems and methods also involve several independent neural networks and neural network models for classification and quantification of each gas, ambient relative humidity and temperature compensation, long-term stability improvement/self-correction or calibration mechanisms, etc. and are intentionally kept modular for continuous improvement as needed. Neural network models are empirically derived from extensive testing in controlled laboratory environments and/or real-world scenarios. Such neural networks (NNs) and NN models may be pre-programmed or may be continuously learning and regenerated by using artificial intelligence (AI) and/or machine learning (ML) routines and subroutines. The NN models may be pre-trained NNs.

In an embodiment, an array comprising one or more first MOx sensors, one or more second MOx sensors, and one or more of a humidity and temperature (RH/T) sensors is used to detect, identify, and quantify volatile organic compounds (VOCs), Ozone (O₃), and Nitrogen Dioxide (NO₂), while at the same time mitigating the cross interference of each gas with the others.

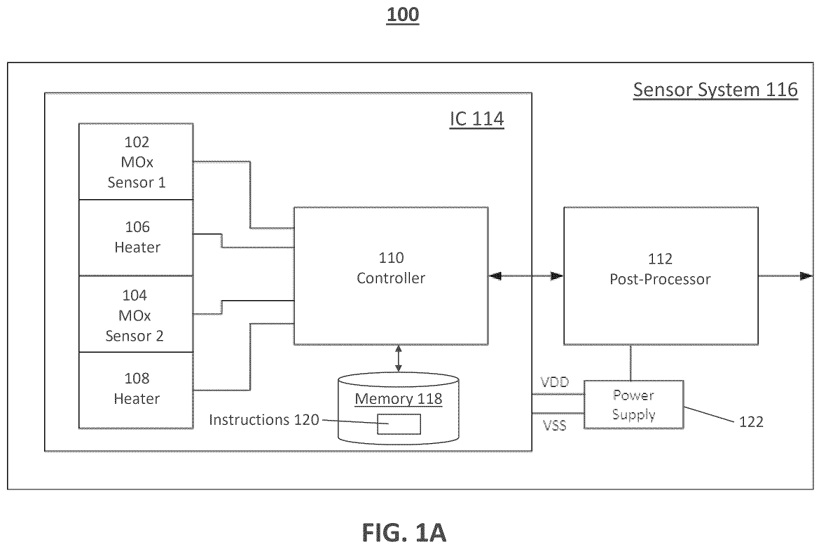

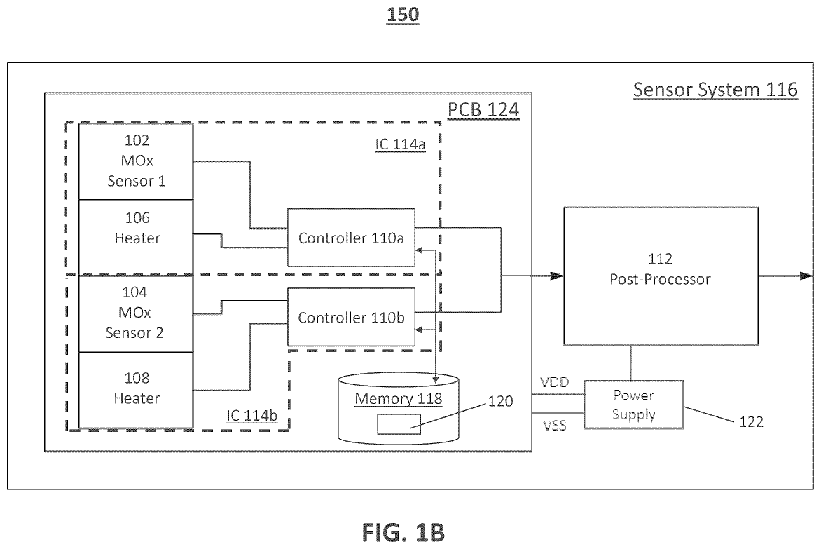

FIG. 1A illustrates a block diagram of a system 100 in which embodiments of the present disclosure may be implemented. One or more first MOx sensors 102 are thermally coupled to heater 106 and one or more second MOx sensors 104 are thermally coupled to heater 108. Heaters 106 and 108 control the operating temperature of the one or more first MOx sensors 102 and the one or more second MOx sensors 104, respectively.

In an embodiment, to provide more accurate measurements, the one or more first MOx sensors 102 and the one or more second MOx sensors 104 may be part of single package or substrate and are thermally coupled to either one or more common heaters or one or more individual heaters 106 and 108. Additionally, both the one or more first MOx sensors 102 and the one or more second MOx sensors 104 and their heaters 106 and 108 respectively may be coupled to one or more controllers 110.

In an embodiment, the one or more first MOx sensors 102 may comprise one or more multi-pixel sensors to provide more accurate measurements from the one or more first MOx sensors 102 and may be able to provide measurements based on the location each pixel is measuring for the one or more first MOx sensors 102. Likewise, in another embodiment, the one or more second MOx sensors 104 may also be one or more multi-pixel sensors to provide more accurate measurements from the one or more second MOx sensors 104 and may be able to provide measurements based on the location each pixel is measuring for the one or more second MOx sensors 104. Additionally, in other embodiments, both the one or more first MOx sensors 102 and the one or more second MOx sensors 104 may each comprise one or more multi-pixel sensors or only one of either the one or more first MOx sensors 102 and the second MOx sensor 104 may comprise one or more multi-pixel sensors.

The one or more first MOx sensors 102 have a conductivity that is sensitive to specific gases, namely volatile organic compounds (VOCs) and O₃. VOCs refer to a family of high vapor pressure and low water-solubility chemicals that often result from manufacturing, combustion, out-gassing, or cleaning materials. These VOCs often present health risks and consequently a great interest in monitoring VOCs to provide a measurement of air quality has developed. VOCs include, for example, industrial solvents, fuel oxygenates, by-products of chlorination in water treatment, and are components of petroleum fuels, hydraulic fluids, paint thinners, dry cleaning agents, and other such products. The conductivity of the one or more first MOx sensors 102 is dependent on the presence of these or similar materials in the sampled gas.

Additionally, the one or more first MOx sensors 102 are thermally coupled to a heater 106 and the operation of the one or more first MOx sensors 102 may be affected by the presence of oxidizing gases, including the presence of O₃. The presence of O₃ can affect the baseline and the accuracy of the detection of VOCs in the air samples. Therefore, prior to any real-time measurement of the detection of VOCs and O₃, a baseline value for the one or more first MOx sensors 102 may be determined, as discussed in further detail below.

As mentioned above, similarly to the one or more first MOx sensors 102, the one or more second MOx sensors 104 are thermally coupled to a heater 108. The one or more second MOx sensors 104 have a conductivity that is configured to detect the presence of O₃ and NO₂ in the air. Similar to the one or more first MOx sensors 102, prior to any real-time measurement of the detection of O₃ and NO₂, a baseline value for the one or more second MOx sensors 104 is determined, as discussed in further detail below.

Both the one or more first MOx sensors 102 and the one or more second MOx sensors 104 and their heaters 106 and 108 respectively are coupled to a controller 110. The controller 110 may include one or more processors and includes a combination of analog and digital circuitry to monitor the conductivity of the one or more first MOx sensors 102 and the one or more second MOx sensors 104 while controlling the temperature of the one or more first MOx sensors 102 and the one or more second MOx sensors 104 through the heaters 106 and 108 respectively. The controller 110 is also coupled to a memory 118 and the memory stores non-transitory computer-readable instructions 120 thereon that when executed by the controller 110 cause the instructions 120 stored in the memory 118 to be performed. The one or more first MOx sensors 102, the one or more second MOx sensors 104, heaters 106 and 108, controller 110, and memory 118 may be part of an integrated circuit (IC) 114. In an embodiment, the IC 114 may be an IC and/or a printed circuit board (PCB) that is part of a sensor system 116. In an embodiment controller 110 and/or the IC 114 may be an application specific integrated circuit (ASIC).

In an embodiment, in addition to the controller 110 including one or more processors, the controller 110 may also include one or more memories 118 that each store their own instructions 120 and the one or more processors that process the data received by the one or more first MOx sensors 102 and the one or more second MOx sensors 104. Further, the controller 110 may provide processed data to a post-processor 112 coupled to the one or more first MOx sensors 102 and the one or more second MOx sensors 104.

As mentioned above, the controller 110 may include one or more memories 118 and one or more processors capable of operating the systems and methods discussed herein. The post-processor 112 may receive the processed data from the controller 110 and may further provide data and instructions that are executable by the controller 110 to control heaters 106 and 108 and measure signals from the one or more first MOx sensors 102 and the one or more second MOx sensors 104 to process the signals from the one or more first MOx sensors 102 and the one or more second MOx sensors 104.

As further illustrated in FIG. 1A, a power supply 122 provides power for the one or more first MOx sensors 102, the one or more second MOx sensors 104, heaters 106 and 108, the controller 110, and the post processor 112. In some embodiments the one or more first MOx sensors 102, the one or more second MOx sensors 104, heaters 106 and 108, controller 110, post processor 112, and power supply 122 may all be components of an overall sensor system 116 that may be mounted on one or more printed circuit boards (PCBs). However, in other embodiments these components may each be on separate PCBs that are each connected to the overall sensor system 116.

In another embodiment, FIG. 1B provides an illustration of an implementation of the system described in FIG. 1A. In this embodiment, sensor system 116 can include two ICs 114a, 114b, where each one of ICs 114a, 114b can include its respective one or more MOx sensors, heater, and controller. By way of example, IC 114a can include the one or more first MOx sensors 102, heater 106 and a controller 110a. IC 114b can include the one or more first MOx sensors 104, heater 108 and a controller 110b. Controllers 110a, 110b can be individual ASICs and can be copies of controller 110 shown in FIG. 1A. ICs 114a, 114b can be mounted on a PCB 124. PCB 124 may include IC 114a, IC 114b and memory 118 (which stores the instructions 120). PCB 124 may be coupled to a post-processor 112 and power supply 122. Additionally, PCB 124, post-processor 112, and power supply 122 may be parts of an overall sensor system 116. Additionally, or alternatively, IC 114a and IC 114b may be coupled to the same controller 110, as described above with respect to FIG. 1A.

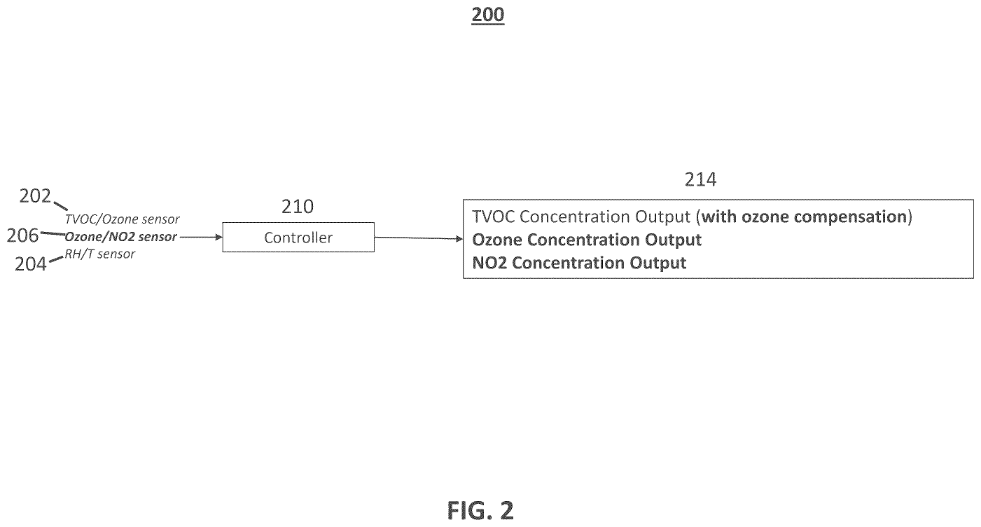

FIG. 2 is a flow chart that illustrates a way to quantify gas in the air, according to an embodiment. This embodiment includes one or more TVOC/O₃ sensors 202, one or more RH/T sensors 204, and one or more O₃/NO₂ sensors 206 (the one or more O₃/NO₂ sensors 206 are not included in the prior art). The controller 110 can receive signals that are output from the one or more TVOC/O₃ sensors 202, the one or more RH/T sensors 204, and the one or more O₃/NO₂ sensors 206 and execute the instructions 120 stored in memory 118 (shown in FIG. 1) to perform a technique that is used to analyze the signals from sensors 202, 204, and 206. In one embodiment, the signals being outputted from sensors 202, 204, and 206 can encode resistance data (e.g., digital data) that quantifies resistances of the sensors 202, 204, and 206. By way of example, controller 110 can be configured to determine the conductivities of the sensors 202, 204, 206 and convert the conductivities into voltages (or voltage signals) that can indicate the resistance of sensors 202, 204, 206. In one embodiment, the conversion can be performed by an analog-to-digital converter (ADC) in controller 110. Controller 110 can be configured to execute instructions 120 and analyze the voltages, in the form of resistance data, from the one or more O₃/NO₂ sensors 206 in addition to the resistance data from the one or more TVOC/O₃ sensors 202 and the one or more RH/T sensors 204. Accordingly, by incorporating the analysis of the resistance data from one or more O₃/NO₂ sensors along with the resistance data from the one or more TVOC/O₃ sensors 202 and the one or more RH/T sensors 204, controller 110 can determine the TVOC concentration output (with O₃ compensation), the O₃ concentration, and the NO₂ concentration 214.

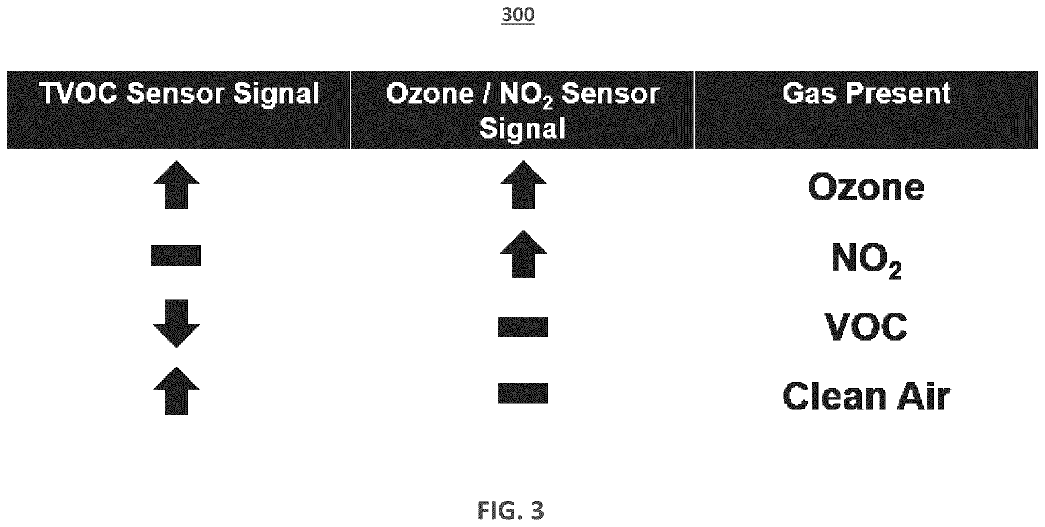

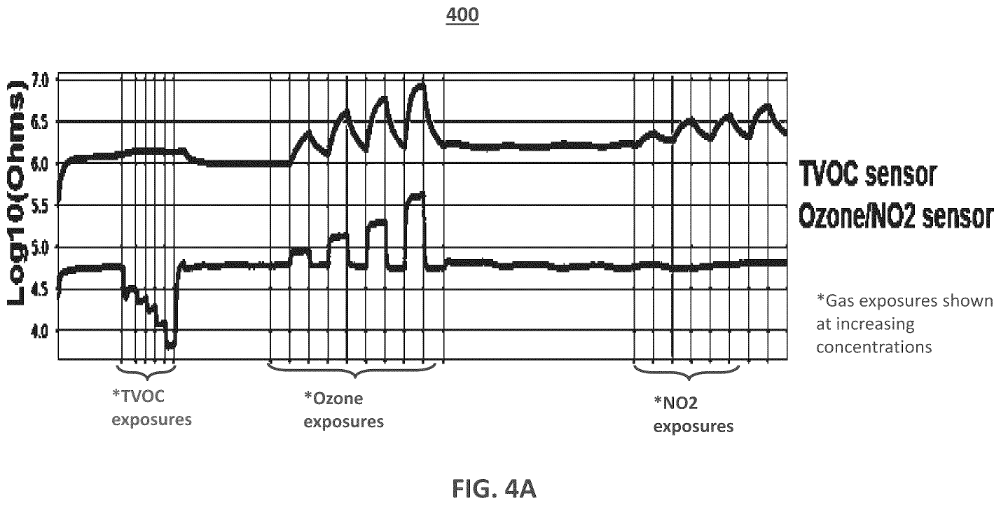

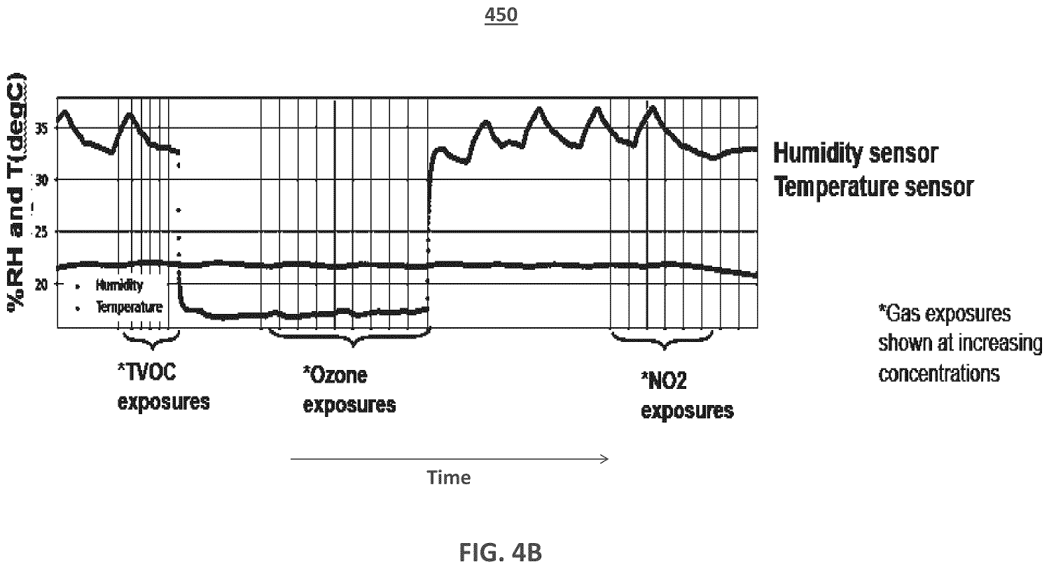

FIG. 3 is a table that illustrates how an increase or decrease of the signals, such as voltages, for both the first MOx sensor signal (i.e., TVOC/O₃ sensor signal) and the second MOx sensor signal (i.e., O₃/NO₂ sensor signal) may determine which gas is present in the air. In one embodiment, controller 110 can be configured to determine a detection result based on the resistance data of the sensors 202, 204, 206. By way of example, controller 110 can map the resistance data of the sensors 202, 204, 206 to time (see FIG. 4) and determine a detection result from the mapping, where the detection result can show variations of the resistance data with respect to time. According to an embodiment of the present disclosure, an increase in the first MOx sensor signal, or resistance data, above a first predetermined range and an increase in the second MOx sensor signal, or resistance data, above a second predetermined range indicates that O₃ is the gas that is present in the air. According to another embodiment of the present disclosure, if the first MOx sensor signal stays within the first predetermined range and the second MOx sensor signal increases above a second predetermined range then NO₂ is the gas that is present. According to yet another embodiment of the present disclosure, a decrease in the first MOx sensor signal, or resistance data, below a first predetermined range and the second MOx sensor signal, or resistance data, staying within a predetermined second range indicates that volatile organic compounds (VOCs) are present in the air. According to still yet another embodiment of the present disclosure, an increase in the first MOx sensor signal, or resistance data, above a first predetermined range and the second MOx sensor signal, or resistance data, staying within a predetermined second range indicates that the air is clean. In one embodiment, resistance data from the sensors can be increasing at a relatively slow pace, thus the first predetermined range can be lowered when compared to situations where the resistance data increases at relatively faster rate. In one embodiment, the first predetermined range and the second predetermined range can be dynamically adjusted using various signal and noise statistics and characteristics of the resistance data previously outputted by the sensors.

FIG. 4A illustrates the typical raw responses (e.g., resistance data) for a TVOC sensor and O₃/NO₂ sensor vs. varying concentrations of TVOC, O₃ and NO₂, according to embodiments of the present disclosure. The raw responses in FIG. 4 can reflect variations of resistance data from sensors 202, 204, 206 when the sensors 202, 204, 206 are exposed to an environment that can include one or more gases within a period of time. As depicted in FIG. 4A, in an embodiment of the present disclosure, when O₃ is present in the air, the output signal, or resistance data, of the TVOC sensor increases and the output signal, or resistance data, of the O₃/NO₂ sensor increases. In other embodiments, the output signal, or resistance data, of either one or both of the TVOC sensor and O₃/NO₂ sensor may be a combined signal value or individual signal values from an array of one or more TVOC sensors and/or one or more O₃/NO₂ sensors.

In response to O₃ being present in the air, the one or more TVOC sensors and/or controller 110 may detect, identify, and quantify the TVOC by using O₃ compensation. Additionally, the one or more O₃/NO₂ sensors may estimate the O₃ concentration because of an O₃ logic check being confirmed (more details about the O₃ logic check are disclosed below).

In contrast and in another embodiment of the present disclosure, in an environment of clean air where there is no O₃ present, the output signals, or resistance data, of the one or more TVOC sensors and the one or more O₃/NO₂ sensors do not vary or have a small drift. In this embodiment, the one or more TVOC sensors may detect, identify, and quantify the TVOC since O₃ compensation is not necessary because O₃ is not present in the air.

Additionally, in a related embodiment, the one or more O₃/NO₂ sensors may estimate the concentration of NO₂ because of the O₃ logic check confirming the absence of O₃ (more details about the O₃ logic check are disclosed below).

FIG. 4B illustrates the typical raw responses for a humidity sensor and a temperature sensor specified as percent humidity (% RH) and degrees Celsius (° C.), according to embodiments of the present disclosure. In some embodiments there may be one or more humidity sensors and/or one or more temperature sensors.

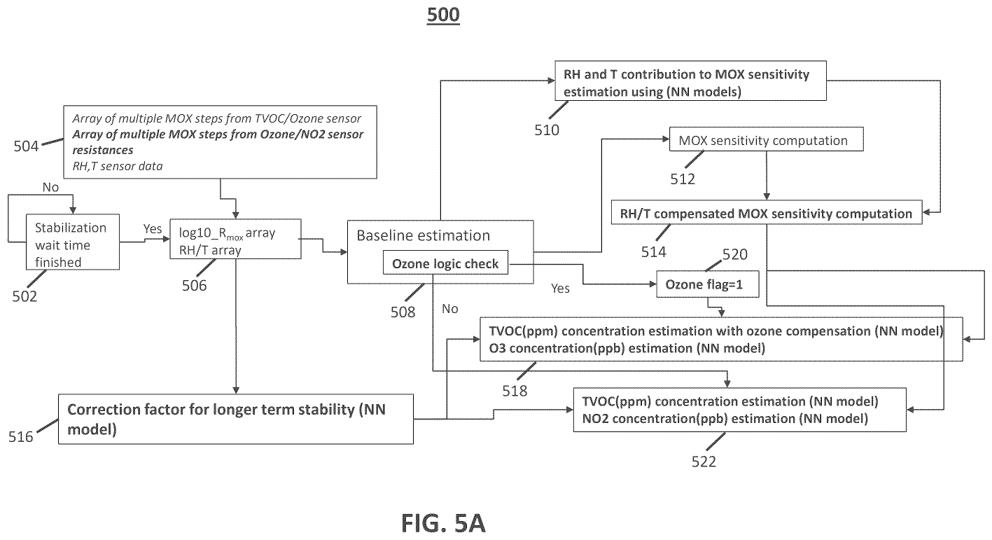

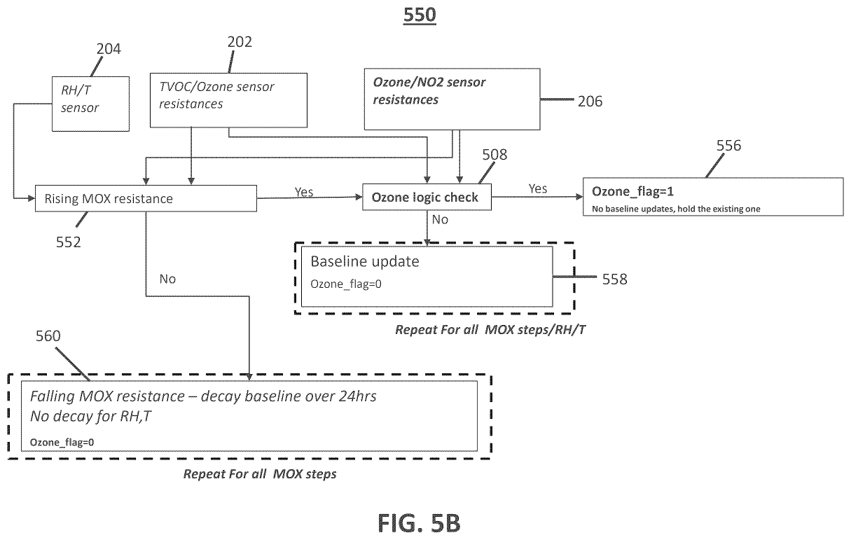

FIGS. 5A and 5B illustrate exemplary flow charts of techniques for dynamically estimating baseline values using one or more MOX sensor resistances and avoiding the influence of Ozone, and for detecting, identifying and quantifying multiple gases in the air, with built-in RH/T compensation, and ozone compensation, according to embodiments of the present disclosure. The steps that are illustrated in both FIGS. 5A and 5B may be repeated for every new data sample.

According to embodiments of the present disclosure, FIG. 5A illustrates an example process that can be performed by controller 110 as a result of executing instructions 120 to determine gases in the air. At step 502 the system waits for stabilization of the sensors and their corresponding signals. At step 504 a result is determined by a combination of an array of multiple MOx steps from the TVOC/O₃ sensor(s), an array of multiple MOx steps from the O₃/NO₂ sensor, and humidity and temperature (RH/T) sensors.

After the stabilization wait time for step 502 is finished and the results from step 504 are determined, the results determined by step 504 are transformed at step 506 by a logarithmic function. The output of the logarithmic function at step is communicated to step 508 for a dynamic baseline estimation, which considers the presence of O₃ and adjusts accordingly. Additionally, the output of the logarithmic function is also communicated to step 516 so that it can be combined with prior results of the logarithmic function to optimize a correction factor for longer term stability for quantifying the presence of certain gases in the air. The correction factor can be provided from step 516 to steps 518, 522.

In an embodiment, the correction factor for longer term stability may be quantified using a neural network (NN) and/or a neural model. Such neural networks (NNs) and NN models may be pre-programmed or may be continuously learning and regenerated by using artificial intelligence (AI) and/or machine learning (ML) routines and subroutines. The NN models may be pre-trained NNs.

At step 508 a baseline estimation for the O₃ concentration is determined and as a result an O₃ logic check determines a “yes” or “no” (e.g., an O₃ flag=1 or 0). The baseline estimation at step 508 is used at step 510 to determine the relative humidity and/or temperature (RH and/or T) contribution of the one or more MOx sensitivities. These contributions are estimated using RH and/or T baseline values and/or latest values as inputs to NN models similar to those mentioned above.

Additionally, at step 512, the one or more MOx sensitivities are computed based on the baseline estimation at step 508. The results determined at step 510 and step 512 are then used as inputs at step 514 in order to determine humidity and temperature compensated MOx sensitivities.

After the humidity and temperature compensated MOx sensitivity computation is determined at step 514 and the correction factor for longer term stability is determined at step 516, the results for each determination are used at step 518 and/or step 522 to determine the TVOC (parts per million (ppm)) concentration with O₃ compensation using an NN model (see above). In one embodiment, estimation and determination of the TVOC concentration in step 518 and/or step 522 can use the correction factor from step 516 to compensate for overall output accuracy.

At step 508, in response to the O₃ logic check determining a “yes,” the O₃ flag is set to 1, and the baseline values of the one or more of the first and the second MOX sensors are used at step 518 in conjunction with the results from step 514 and step 516 (see above) to determine the O₃ concentration (parts per billion (ppb)) using an NN model (see above). In one embodiment, estimation and determination of the O₃ concentration in step 518 can use the correction factor from step 516 to compensate for overall output accuracy.

In contrast, in response to the O₃ logic check determines a “no” (e.g., the O₃ flag=0) at step 508, the baseline values of the one or more of the first and the second MOX sensors are used at step 522 in conjunction with the results from step 514 and step 516 (see above) to determine NO₂ concentration (parts per billion (ppb)) using a NN model (see above). In one embodiment, estimation and determination of the NO₂ concentration in step 522 can use the correction factor from step 516 to compensate for overall output accuracy.

FIG. 5B illustrates another exemplary flow chart of the dynamic baseline estimation process with O₃ awareness, that can be performed by controller 110 as a part of executing instructions 120 for detecting, identifying, and quantifying multiple gases in the air, according to embodiments of the present disclosure. To begin the flow chart illustrated by FIG. 5B, at step 552 a sensor measurement sample array may be determined by one or more RH/T sensors 204, one or more TVOC/O₃ sensor resistances 202, and one or more O₃/NO₂ sensor resistances 206. In an embodiment, the one or more RH/T sensors 204, the one or more TVOC/O₃ sensors 202, and the one or more O₃/NO₂ sensors 206 may be arranged in one or more arrays in combination or not in combination with each other.

At step 552 it may be determined if the MOx resistance is rising. If it is determined that the MOx resistance is rising, an O₃ logic check is conducted in step 508 based on resistances of the one or more TVOC/O₃ sensors 202 and resistances of the one or more O₃/NO₂ sensor 206. In response to determining that the O₃ logic check in step 508 is a “yes”, the O₃ flag 556 is set to 1. As a result of the O₃ flag 556 being set to 1, baseline updates are cancelled or paused, and the current baseline values continue to be used.

On the other hand, in response to the O₃ logic check in step 508 being a “no”, the O₃ flag 558 is set to 0 and the baseline updates continue to repeat for the one or more RH/T sensors 204, the one or more TVOC/O₃ sensor resistances 202, and the one or more O₃/NO₂ sensor resistances 206.

In response to step 552 determining that the MOx resistance is not rising, the flow chart moves to step 560 where the O₃ flag is set to 0 and the baseline is decayed over 24 hours with the exception of any decay for the RH/T (i.e., no decay for the RH/T is recorded). Baseline decay may be over a period shorter or longer than a predetermined time, such as 24 hours. Baseline decay parameter can also be dynamically adjusted based on current or previous sensor signal and/or noise characteristics and/or previous outputs.

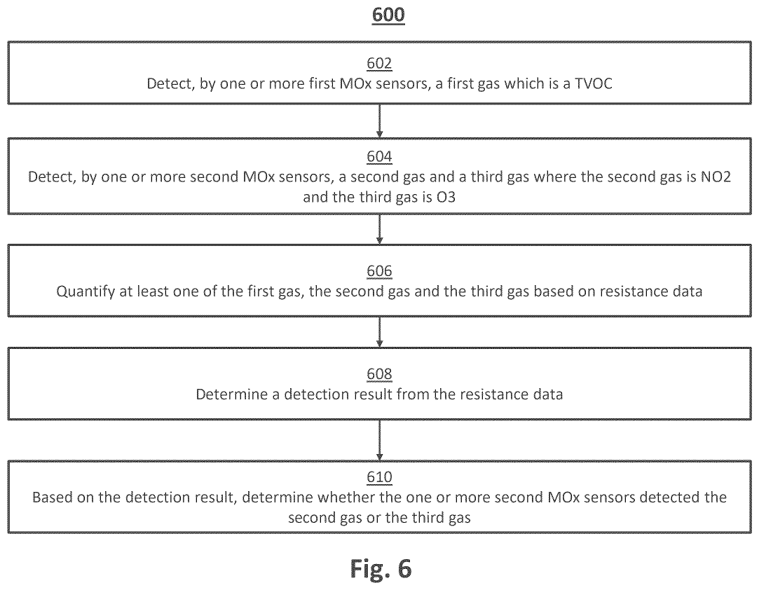

FIG. 6 is a flow diagram 600 of method steps for detecting, identifying and quantifying gas in the air, according to embodiments of the present disclosure. In embodiments, the method 600 may be performed by a sensor system including at least one sensors and one or more processors. The one or more processors may be in communication with one or more memories that store the instructions to be performed by the one or more processors to execute at least some of the steps for quantifying gas in the air, as set forth in FIG. 6.

As shown in FIG. 6., the method begins at step 602, where one or more first metal-oxide (MOx) sensors are configured to detect a first gas. The first gas may be a total volatile organic compound (TVOC). In other embodiments, the first gas may be another gas that is different from a TVOC.

Additionally, in this and other embodiments, the one or more humidity and temperature (RH/T) sensors may be configured to detect humidity and temperature. Based on the detected humidity and temperature from the RH/T sensors, the one or more processors may quantify humidity and temperature.

At step 604, one or more second MOx sensors may be configured to detect at least one of a second gas and a third gas, where the second gas is nitrogen dioxide (NO₂) and the third gas is Ozone (O₃). In other embodiments, the second gas may be O₃ and the third gas may be NO₂ and may be quantified as such.

Further, in this and other embodiments, the one or more first MOx sensors, the one or more second MOx sensors, and the one or more RH/T sensors may be organized in one or more sensor arrays. Each of the one or more sensor arrays may comprise one of each of the one or more first MOx sensors, the one or more second MOx sensors, and the one or more RH/T sensors.

At step 608, the one or more processors can receive resistance data representing resistances of the one or more first MOx sensors and the one of more second MOx sensors. The one or more processors can quantify the first gas, the second gas and the third gas based on the resistance data.

At step 608, the one or more processors may determine a detection result from the resistance data.

At step 610, the one or more processors may determine, based on the detection result from step 608, whether the one or more second MOx sensors detected the second gas or the third gas.

In response to an increase in the detection result from the resistance data of the one or more first MOx sensors increasing above a first predetermined range and the resistance data of the one or more second MOx sensors increasing above a second predetermined range, the one or more processors may determine that O₃ is present.

In response to an increase in the detection result from the one or more first MOx sensors staying within a first predetermined range and the resistance data of the one or more second MOx sensors increasing above a second predetermined range the one or more processors may determine, that NO₂ is present.

In response to an increase in the detection result from the one or more first MOx sensors decreasing below a first predetermined range and the resistance data of the one or more second MOx sensors staying within a second predetermined range, the one or more processors may determine that volatile organic compounds (VOCs) are present.

In response to an increase in the detection result from the one or more first MOx sensors increasing above a first predetermined range and the resistance data of the one or more second MOx sensors staying within a second predetermined range, the one or more processors may determine that the air is clean.

Additionally, the one or more processors may determine baseline values for the one or more first MOx sensors and the one or more second MOx sensors, and the one or more RH and/or T sensors.

Further, the one or more processors may compare the previously determined baseline values for the one or more first MOx sensors and the one or more second MOx sensors to the detection result from the resistance data received from the one or more first MOx sensors and the one or more second MOx sensors.

Still further, the one or more processors may detect, identify and quantify the amount of TVOC, O₃, and NO₂, in response to the detection result being compared to the previously determined baseline values for the one or more first MOx sensors and the one or more second MOx sensors.

The above detailed description is provided to illustrate specific embodiments of the present invention and is not intended to be limiting. Numerous variations and modifications within the scope of the present invention are possible. The present invention is set forth in the following claims.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The disclosed embodiments of the present invention have been presented for purposes of illustration and description but are not intended to be exhaustive or limited to the invention in the forms disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

What is claimed is:

1. An apparatus comprising:
one or more first metal-oxide (MOx) sensors configured to detect at least one of a first gas and a third gas, wherein the first gas is a total volatile organic compound (TVOC) and the third gas is Ozone (03);
one or more second MOx sensors configured to detect at least one of a second gas and the third gas, wherein the second gas is nitrogen dioxide (NO₂); and
at least one processor configured to:
receive resistance data from the one or more first MOx sensors and the one or more second MOx sensors, wherein the resistance data is based on detection of the first gas, the second gas and the third gas by the one or more first MOx sensors and the one or more second MOx sensors;
quantify at least one of the first gas, the second gas, and the third gas based on the resistance data;
determine a detection result from the resistance data of the first gas, the second gas, and the third gas; and
based on the detection result, determine whether the one or more first MOx sensors and the one or more second MOx sensors detected a presence or absence of the third gas.

2. The apparatus according to claim 1, further comprising one or more humidity and temperature (RH/T) sensors to detect humidity and temperature, and the at least one processor configured to:
quantify the humidity and temperature detected by the one or more RH/T sensors;
determine a compensation value based on the quantified humidity and temperature; and
quantify at least one of the first gas, the second gas, and the third gas based on the resistance data and the compensation value.

3. The apparatus according to claim 2, wherein the one or more first MOx sensors, the one or more second MOx sensors, and the one or more RH/T sensors are organized in one or more sensor arrays, and wherein each of the one or more sensor arrays comprise one of each the first MOx sensor, the second MOx sensor, and the RH/T sensor.

4. The apparatus according to claim 1, wherein the at least one processor is further configured to:
in response to an increase in the detection result from the resistance data of the one or more first MOx sensors increasing above a first predetermined range and the resistance data of the one or more second MOx sensors increasing above a second predetermined range, determine that 03 is present.

5. The apparatus according to claim 1, wherein the at least one processor is further configured to:
in response to an increase in the detection result from the resistance data of the one or more first MOx sensors staying within a first predetermined range and the resistance data of the one or more second MOx sensors increasing above a second predetermined range, determine that NO₂ is present.

6. The apparatus according to claim 1, wherein the at least one processor is further configured to
in response to an increase in the detection result from the resistance data from the one or more first MOx sensors decreasing below a first predetermined range and the resistance data of the one or more second MOx sensors staying within a second predetermined range, determine that volatile organic compounds (VOCs) are present.

7. The apparatus according to claim 1, wherein the at least one processor is further configured to:
in response to an increase in the detection result from the resistance data of the one or more first MOx sensors increasing above a first predetermined range and the resistance data of the one or more second MOx sensors staying within a second predetermined range, determine that the third gas is absent.

8. The apparatus according to claim 1, wherein the at least one processor is further configured to:
determine baseline values for the one or more first MOx sensors and the one or more second MOx sensors.

9. The apparatus according to claim 8, wherein the at least one processor is further configured to:
compare the determined baseline values for the one or more first MOx sensors and the one or more second MOx sensors to the detection result from the resistance data received from the one or more first MOx sensors and the resistance data received from the one or more second MOx sensors.

10. The apparatus according to claim 9, wherein the at least one processor is configured to:
quantify amounts of TVOC, O₃, and NO₂ in response to the detection result being compared to the determined baseline values for the one or more first MOx sensors and the one or more second MOx sensors.

11. A method comprising:
detecting, by one or more first metal-oxide (MOx) sensors in a sensor system, at least one of a first gas and a third gas, wherein the first gas is a total volatile organic compound (TVOC) and the third gas is Ozone (O₃);
detecting, by one or more second MOx sensors in the sensor system, at least one of a second gas and a third gas, wherein the second gas is nitrogen dioxide (NO₂);
receiving, by one or more processors of the sensor system, resistance data from the one or more first MOx sensors and the one or more second MOx sensors, wherein the resistance data is based on detection of the first gas, the second gas and the third gas by the one or more first MOx sensors and the one or more second MOx sensors;
quantifying, by the one or more processors of the sensor system, at least one of the first gas, the second gas, and the third gas based on the resistance data;
determining, by the one or more processors of the sensor system, a detection result from resistance data of the first gas, the second gas, and the third gas; and
based on the detection result, determining, by the one or more processors of the sensor system, whether the one or more first MOx sensors and the one or more second MOx sensors detected a presence or absence of the third gas.

12. The method according to claim 11, further comprising:
detecting, by one or more humidity and temperature (RH/T) sensors of the sensor system, humidity and temperature;
quantifying, by the one or more processors, the humidity and temperature detected by the one or more RH/T sensors;
determining, by the one or more processors, a compensation value based on the quantified humidity and temperature; and
quantifying, by the one or more processors, at least one of the first gas, the second gas, and the third gas based on the resistance data and the compensation value.

13. The method according to claim 12, wherein the one or more first MOx sensors, the one or more second MOx sensors, and the one or more RH/T sensors are organized in one or more sensor arrays, and wherein each of the one or more sensor arrays comprise one of each of the one or more first MOx sensors, the one or more second MOx sensors, and the one or more RH/T sensors.

14. The method according to claim 11, further comprising:
in response to an increase in the detection result from the resistance data of the one or more first MOx sensors increasing above a first predetermined range and the resistance data of the one or more second MOx sensors increasing above a second predetermined range, determining, by the one or more processors, that O₃ is present.

15. The method according to claim 11, further comprising:
in response to an increase in the detection result from the one or more first MOx sensors staying within a first predetermined range and the resistance data of the one or more second MOx sensors increasing above a second predetermined range, determining, by the one or more processors, that NO₂ is present.

16. The method according to claim 11, further comprising:
in response to an increase in the detection result from the one or more first MOx sensors decreasing below a first predetermined range and the resistance data of the one or more second MOx sensors staying within a second predetermined range, determining, by the one or more processors, that volatile organic compounds (VOCs) are present.

17. The method according to claim 11, further comprising:
in response to an increase in the detection result from the one or more first MOx sensors increasing above a first predetermined range and the resistance data of the one or more second MOx sensors staying within a second predetermined range, determining, by the one or more processors that the third gas is absent.

18. The method according to claim 11, further comprising:
previously determining, by the one or more processors, baseline values for the one or more first MOx sensors and the one or more second MOx sensors; and
comparing, by the one or more processors, the previously determined baseline values for the one or more first MOx sensors and the one or more second MOx sensors to the detection result from the resistance data received from the one or more first MOx sensors and the one or more second MOx sensors.

19. The method according to claim 18, further comprising:
quantifying, by the one or more processors, amounts of TVOC, O₃, and NO₂ in response to the detection result being compared to the previously determined baseline values for the one or more first MOx sensors and the one or more second MOx sensors.

20. At least one non-transitory computer-readable storage medium configured to store a computer-executable program comprising instructions which, when executed by a processor, causes the processor to at least perform:
receiving resistance data from the one or more first MOx sensors and the one or more second MOx sensors, wherein the resistance data is based on a detection of at least one of a first gas and a third gas by one or more first MOx sensors and a detection of at least one of a second gas and the third gas by one or more second MOx sensors, the first gas is a total volatile organic compound (TVOC), the second gas is nitrogen dioxide (NO₂) and the third gas is Ozone (O₃);
quantifying at least one of the first gas, the second gas, and the third gas based on the resistance data;
determining a detection result from the resistance data of the first gas, the second gas, and the third gas; and
based on the detection result, determining whether the one or more first MOx sensors and the one or more second MOx sensors detected a presence or absence of the third gas.
